(19) United States
(12) Patent Application Publication
(10) Pub. No.: US 2026/0320997 A1
(43) Pub. Date: Oct. 15, 2026

(54) DIGITAL PROFILING OF FINE-GRAINED SPATIAL TRANSCRIPTOMICS FROM IMAGES

(71) Applicant: NEC Laboratories America, Inc., Princeton, NJ (US)

(72) Inventors: Jonathan Warrell, Princeton, NJ (US); Eric Cosatto, Red Bank, NJ (US); Renqiang Min, Princeton, NJ (US); Tianci Song, Eagen, MN (US)

(21) Appl. No.: 19/462,040

(22) Filed: Jan. 28, 2026

Related U.S. Application Data
(60) Provisional application No. 63/751,977, filed on Jan. 31, 2025.

(51) Int. Cl. G06V 10/86; G06N 3/042; G06N 3/0455; G06V 10/762; G06V 10/82; G06V 20/69; G16B 25/10; G16B 45/00

(52) U.S. Cl. CPC G06V 10/86; G06N 3/042; G06N 3/0455; G06V 10/762; G06V 10/82; G06V 20/695; G06V 20/698; G16B 25/10; G16B 45/00; G06V 2201/03

(57) ABSTRACT

Systems and methods for digital profiling of fine-grained spatial transcriptomics from images. Image data can be converted into a spatial compartment graph that represents vectors of image features extracted from the images. Imputed transcriptomics can be determined with an adaptive spatial compartment graph transformer network (ascGTN) by utilizing the spatial compartment graph. An imputed spatial marker can be learned with the ascGTN based on the imputed transcriptomics. Future events related to downstream tasks can be detected based on the imputed spatial marker and resolve issues related to the future events.

### RELATED APPLICATION INFORMATION

This application claims priority to U.S. Provisional App. No. 63/751,977, filed on Jan. 31, 2025, incorporated herein by reference in its entirety.

### BACKGROUND

### Technical Field

The present invention relates to image processing with artificial intelligence (AI), and more particularly digital profiling of fine-grained spatial transcriptomics from images.

### Description of the Related Art

Artificial intelligence can be used to process images and learn features about the images. Spatial features around a monitored entity within the image can be determined and processed for various fields. For example, in the medical field, cellular interactions can be monitored based on the spatial features around a particular cell. Additionally, in a traffic scene, interactions between vehicles can be monitored based on the spatial features around a particular vehicle.

### SUMMARY

According to an aspect of the present invention, a method is provided including, converting image data into a spatial compartment graph that represents vectors of image features extracted from the images, determining imputed transcriptomics with an adaptive spatial compartment graph transformer network (ascGTN) by utilizing the spatial compartment graph, learning an imputed spatial marker with the ascGTN based on the imputed transcriptomics, and detecting future events related to downstream tasks based on the imputed spatial marker and resolving issues related to the future events.

According to another aspect of the present invention, a system is provided, including, a memory device, one or more processor devices operatively coupled with the memory device to perform operations including, converting image data into a spatial compartment graph that represents vectors of image features extracted from the images, determining imputed transcriptomics with an adaptive spatial compartment graph transformer network (ascGTN) by utilizing the spatial compartment graph, learning an imputed spatial marker with the ascGTN based on the imputed transcriptomics, and detecting future events related to downstream tasks based on the imputed spatial marker and resolving issues related to the future events.

According to yet another aspect of the present invention, a non-transitory computer program product is provided including a computer-readable storage medium including a program code, wherein the program code when executed on a computer causes the computer to perform operations including, converting image data into a spatial compartment graph that represents vectors of image features extracted from the images, determining imputed transcriptomics with an adaptive spatial compartment graph transformer network (ascGTN) by utilizing the spatial compartment graph, learning an imputed spatial marker with the ascGTN based on the imputed transcriptomics, and detecting future events related to downstream tasks based on the imputed spatial marker and resolving issues related to the future events.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram that shows a computer system for digital profiling of fine-grained spatial transcriptomics from images, in accordance with one embodiment of the present invention;

FIG. 2 is a block diagram that shows a computer system for digital profiling of fine-grained spatial transcriptomics from images, in accordance with an embodiment of the present invention;

FIG. 3 is a block diagram that shows components of a computer system for digital profiling of fine-grained spatial transcriptomics from images, in accordance with an embodiment of the present invention;

FIG. 4 is a block diagram that shows components of an adaptive spatial compartment graph transformer network for digital profiling of fine-grained spatial transcriptomics from images, in accordance with an embodiment of the present invention;

FIG. 5 is a block diagram that shows a neural network for digital profiling of fine-grained spatial transcriptomics from images, in accordance with an embodiment of the present invention; and

FIG. 6 is a flow diagram that shows a high-level overview of a method for digital profiling of fine-grained spatial transcriptomics from images, in accordance with an embodiment of the present invention.

### DETAILED DESCRIPTION OF PREFERRED EMBODIMENTS

In accordance with embodiments of the present invention, systems and methods are provided for digital profiling of fine-grained spatial transcriptomics from images.

In the embodiments, image data can be converted into a spatial compartment graph that represents vectors of image features extracted from the images. Imputed transcriptomics can be determined with an adaptive spatial compartment graph transformer network (ascGTN) by utilizing the spatial compartment graph. An imputed spatial marker can be learned with the ascGTN based on the imputed transcriptomics. Future events related to downstream tasks can be detected based on the imputed spatial marker and resolve issues related to the future events.

Spatial transcriptomics provides an informative data source for how cancer patients will respond to treatments, such as immunotherapy targeting particular pathways. Techniques such as Digital Spatial Profiling (DSP) allow cell-type specific information to be captured in the spatial profiling of gene expression, allowing important information to be captured about the tumor microenvironment (TME) relevant for biomarker discovery. However, techniques such as DSP are highly expensive, and cannot be performed routinely for patient screening. In contrast, Hematoxylin and Eosin (H&E) images are routinely captured and analyzed by Pathologists. The general problem of imputing gene expression from H&E images is thus important for tasks such as identifying and evaluating biomarkers. More particularly imputing spatial gene expression at cell-type specific resolution is important for increasing the accuracy of such biomarkers, and revealing detailed information about a tumor's development for personalized medicine. Further, it is important to be able to use data of multiple types in learning predictors for spatial gene expression imputation, since the quantities of spatial transcriptomics and cell-type specific spatial transcriptomics with paired H&E images available for training are small, while there are large quantities of bulk transcriptomics paired with H&E images from public sources such as TCGA.

However, there are issues with determining spatial transcriptomics from images:
Effort: Additional efforts (e.g. collecting separate tissue samples) are required to profile transcriptomics, and current workflows are not designed to incorporate these in many clinical tests.
Cost: Despite sustained decrease in cost, transcriptomics profiling technologies are still not cost-effective to be integrated in the routine diagnostics.
Time: Time-consuming (more than one week) and labor-intensive procedures pose additional difficulties in applying transcriptomics profiling technologies to clinical routines.
Spatial Resolution: Fine-grained spatial transcriptomics (cellular or subcellular) are important to probe tumor micro-environment (TME) interactions, it is almost impossible to use in clinical studies due to its prohibitive cost.

Previous methods have used tile-based methods for imputing spatial transcriptomics from H&E images. These methods use features from image tiles to impute bulk tile expression, and therefore cannot impute cell-type specific spatial expression patterns. Moreover, these methods do not consider the spatial relationships between tiles, and do not consider the interaction between different cell-types within a tile. Further, although bulk TCGA data can be used during training, none of the methods are designed to utilize more informative DSP data.

Previous approaches have shown that it is possible to learn informative biomarkers directly from DSP data, which outperform bulk biomarkers. However, such work has not incorporated imputed spatial transcriptomics during training, which has the potential to increase the robustness of the biomarkers learned by including patients for which H&E image data is available but no DSP data during training. Additionally, other works do not allow cell-type specific spatial expression to be imputed, and cannot use multiple datatypes (bulk, spatial, DSP) during training.

The present embodiments resolve the aforementioned issues by training an adaptive spatial compartment Graph Transformer Network (ascGTN) architecture. The present embodiments use the ascGTN architecture to impute cell-type specific spatial transcriptomics data from H&E pathology images. The system can be trained using transcriptomics data of various types, including bulk transcriptomics, tile-level spatial transcriptomics, and cell-type specific spatial transcriptomics, including Digital Spatial Profiling (DSP) data.

Due to its limited footprint, the present embodiments can be embedded in a larger system to learn spatial gene expression biomarkers for patient responses to immunotherapy (and other treatments), allowing both DSP and imaging data to be used in the biomarker discovery process. The present embodiments reduce the computational resource cost (e.g., memory, processing, etc.) of determining fine-grained features such as spatial transcriptomics from images.

The present embodiments can be used for broader purposes, including imputing spatial omics of other kinds (e.g. ATAC-seq, proteomics) when available, imputing data from H&E images to study cell-type interactions in cancer, or imputing data from H&E images to study tumor microenvironment subtypes in cancer.

Embodiments described herein can be entirely hardware, entirely software or including both hardware and software elements. In a preferred embodiment, the present invention is implemented in software, which includes but is not limited to firmware, resident software, microcode, etc.

Embodiments can include a computer program product accessible from a computer-usable or computer-readable medium providing program code for use by or in connection with a computer or any instruction execution system. A computer-usable or computer readable medium can include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium can include a computer-readable storage medium such as a semiconductor or solid state memory, magnetic tape, a removable computer diskette, a random access memory (RAM), a read-only memory (ROM), a rigid magnetic disk and an optical disk, etc.

Each computer program can be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system can also be considered to be embodied in a computer-readable storage medium, configured with a computer program, where the storage medium so configured causes a computer to operate in a specific and predefined manner to perform the functions described herein.

A data processing system suitable for storing and/or executing program code can include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) can be coupled to the system either directly or through intervening I/O controllers.

Network adapters can also be coupled to the system to enable the data processing system to become coupled to other data processing systems or remote printers or storage devices through intervening private or public networks. Modems, cable modem and Ethernet cards are just a few of the currently available types of network adapters.

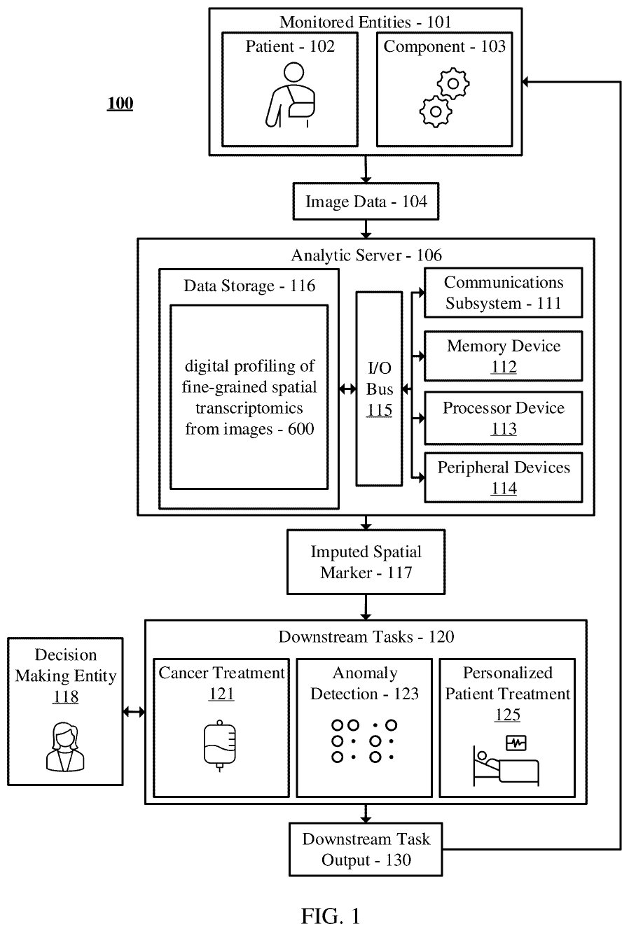

Referring now in detail to the figures in which like numerals represent the same or similar elements and initially to FIG. 1, a block diagram shows a computer system for digital profiling of fine-grained spatial transcriptomics from images, in accordance with one embodiment of the present invention.

In an embodiment using a system 100, monitored entities 101 can include patient 102 and component 103. The monitored entities 140 can generate image data 104. such as image/video, texts, audio, etc. The image data 104 can be transmitted to an analytic server 106 that can implement digital profiling of fine-grained spatial transcriptomics from images 600. The analytic server 106 can obtain an imputed spatial marker 117 that can be utilized to perform downstream tasks 120.

The downstream tasks 120 can include cancer treatment 121, anomaly detection 123, and personalized patient treatment 125. The analytic server 106 can generate a corrective action for the downstream tasks 120 to be sent to respective computing systems for the monitored entities 140 through a network.

In cancer treatment 121, imputed spatial marker 117 can be identified and processed to target cancer cells, determine effect of a cancer treatment, determine response of a patient to the cancer. In an embodiment, the imputed spatial marker 117 can be utilized to assist the decision making of the decision-making entity 118 regarding the cancer treatment 121 of the patient 102 through autonomous decision making. In another embodiment, the imputed spatial marker 117 can be engineered as antibodies that can include T-cells. T-cells can recognize antigens (e.g., MHC-peptide) on abnormal cells (e.g., cancer cells) and can be expressed as TCRs. The TCR can bind to the antigen and the T-cell can release toxic chemicals that can destroy the antigens. The cancer treatment 121 can be provided intravenously, orally, or with other appropriate administration route. Examples of cancer treatment 121 can include chimeric antigen receptor (CAR) T-cell therapy (e.g., Tisagenlecluecel), CAR natural killer (NK) cell therapy, etc.

In anomaly detection 123, image data 104 (e.g., system logs, test cases, hardware status images, etc.) related to the component 103 (e.g., request server of a distributed computing application) can be processed by the analytic server 106 to detect system anomalies (e.g., abnormal increase in requests for a server, abnormal dip in storage resources, abnormal increase in processing power consumption, etc.). With the analytic server 106, autonomous system maintenance can be performed on the component 103 such as stopping/starting component, restarting component, adding bandwidth, blocking packets from an identified internet protocol (IP) address to resolve malicious attacks, restarting hardware, etc. In another embodiment, the decision-making entity 118 (e.g., maintenance professional) responsible for the component 103 can be notified of the detected anomalies to help the decision-making process of the decision-making entity 118 and verify the autonomous system maintenance performed on the component 103 through automated decision making.

In personalized patient treatment 125, the imputed spatial marker 117 can be identified and processed to target the illness of patient 102. In an embodiment, the imputed spatial marker 117 can be utilized to assist the decision making of the decision-making entity 118 regarding the personalized patient treatment 125 of the patient 102 through autonomous decision making. The imputed spatial marker 117 can be engineered as antibodies that can include T-cells. T-cells can recognize antigens (e.g., pMHC) on abnormal cells that causes the illness of patient 102 (e.g., abnormal cells caused by an abnormal mutation in healthy cells) and can be expressed as TCRs. The TCR can bind to the antigen and the T-cell can release toxic chemicals that can destroy the antigens. The personalized patient treatment 125 can be provided to the patient 102 intravenously, orally, or with other appropriate administration route. Examples of personalized patient treatment 125 can include chimeric antigen receptor (CAR) T-cell therapy (e.g., Tisagenlecluecel), gene therapy drug for atopic dermatitis (e.g., abrocitinib), gene therapy drug for hemolytic anema (e.g., mitapivat) etc.

Other downstream tasks and practical applications are contemplated.

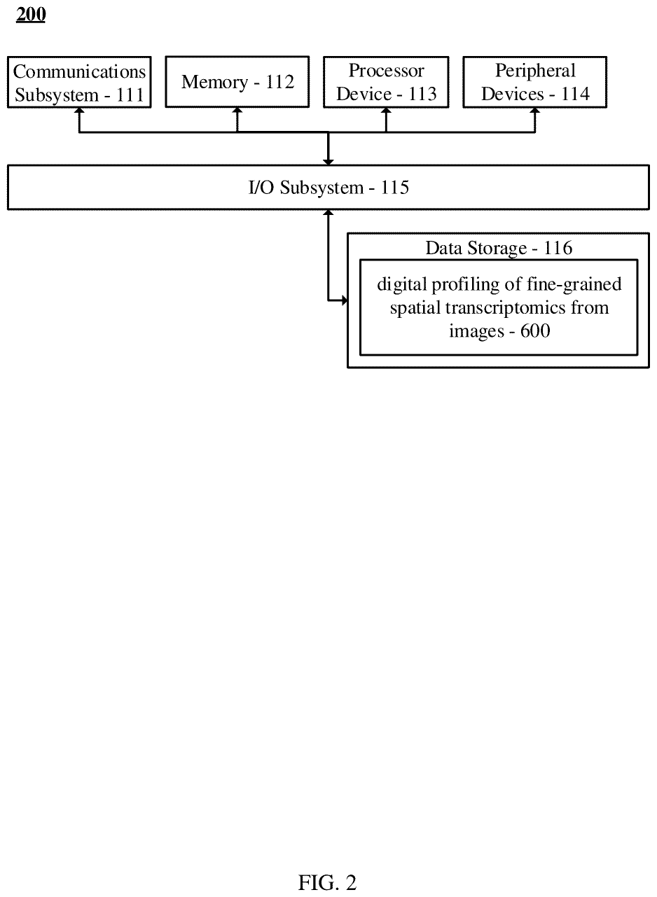

The analytic server 106 can include a processor device 113, data storage device 116, memory 112, communications subsystem 111, peripheral devices 114, and input/output (I/O) bus 115. The analytic server 106 is an implementation of a computer system. Other implementations are contemplated. The computer system is shown in more detail in FIG. 2.

Referring now to FIG. 2, a block diagram shows a computer system for digital profiling of fine-grained spatial transcriptomics from images, in accordance with an embodiment of the present invention.

The computing device 200 illustratively includes the processor device 113, an input/output (I/O) subsystem 190, a memory 112, a data storage device 116, and a communications subsystem 111, and/or other components and devices commonly found in a server or similar computing device. The computing device 200 can include other or additional components, such as those commonly found in a server computer (e.g., various input/output devices), in other embodiments. Additionally, in some embodiments, one or more of the illustrative components can be incorporated in, or otherwise form a portion of, another component. For example, the memory 112, or portions thereof, can be incorporated in the processor device 113 in some embodiments.

The processor device 113 can be embodied as any type of processor capable of performing the functions described herein. The processor device 113 can be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 112 can be embodied as any type of volatile or non-volatile memory or data storage capable of performing the functions described herein. In operation, the memory 112 can store various data and software employed during operation of the computing device 200, such as operating systems, applications, programs, libraries, and drivers. The memory 112 is communicatively coupled to the processor device 113 via the I/O subsystem 115, which can be embodied as circuitry and/or components to facilitate input/output operations with the processor device 113, the memory 112, and other components of the computing device 200. For example, the I/O subsystem 115 can be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 115 can form a portion of a system-on-a-chip (SOC) and be incorporated, along with the processor device 113, the memory 112, and other components of the computing device 200, on a single integrated circuit chip.

The data storage device 116 can be embodied as any type of device or devices configured for short-term or long-term storage of data such as, for example, memory devices and circuits, memory cards, hard disk drives, solid state drives, or other data storage devices. The data storage device 116 can store program code for digital profiling of fine-grained spatial transcriptomics from images 600. Any or all of these program code blocks can be included in a given computing system.

The communications subsystem 111 of the computing device 200 can be embodied as any network interface controller or other communication circuit, device, or collection thereof, capable of enabling communications between the computing device 200 and other remote devices over a network. The communications subsystem 111 can be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 200 can also include one or more peripheral devices 114. The peripheral devices 114 can include any number of additional input/output devices, interface devices, and/or other peripheral devices. For example, in some embodiments, the peripheral devices 114 can include a display, touch screen, graphics circuitry, keyboard, mouse, speaker system, microphone, network interface, and/or other input/output devices, interface devices, GPS, camera, and/or other peripheral devices.

Of course, the computing device 200 can also include other elements (not shown), as readily contemplated by one of skill in the art, as well as omit certain elements. For example, various other sensors, input devices, and/or output devices can be included in computing device 200, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 200 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

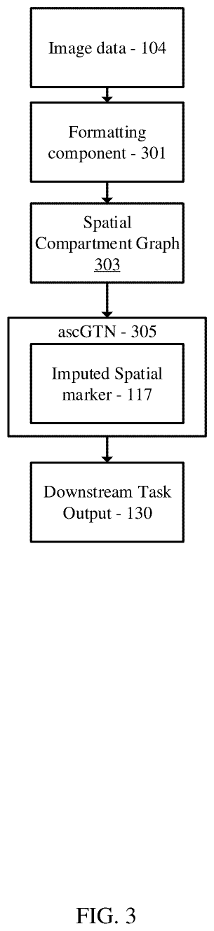

Referring now to FIG. 3, a block diagram shows components of a computer system for digital profiling of fine-grained spatial transcriptomics from images, in accordance with an embodiment of the present invention.

In an embodiment, image data 104 obtained from monitored entities 101 can be processed to generate downstream task output 130. To generate the downstream task output 130, the image data 104 can be processed by a formatting component 301 into a spatial compartment graph 303. The spatial compartment graph 303 can be processed by an adaptive spatial compartment graph transformer network (ascGTN) 305 to determine an imputed transcriptomics 306 based on the spatial compartment graph 303. The imputed transcriptomics 306 can then be utilized to learn an imputed spatial marker 117. The imputed spatial marker 117 can be processed for downstream tasks 120 by utilizing the ascGTN 305 to generate the downstream task output 130.

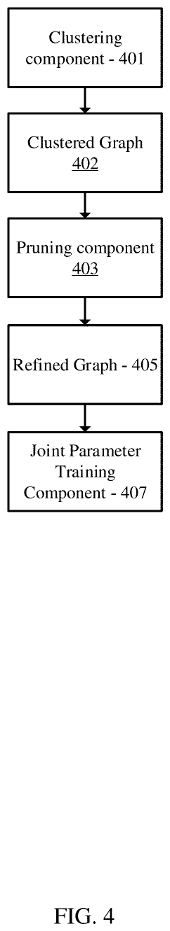

Referring now to FIG. 4, a block diagram shows components of an adaptive spatial compartment graph transformer network for digital profiling of fine-grained spatial transcriptomics from images, in accordance with an embodiment of the present invention.

In an embodiment, ascGTN 305 can include a clustering component 401, a pruning component 403, and a joint parameter training component 407. The clustering component 401 can cluster meta-features from the spatial compartment graph 303 for corresponding weights of the ascGTN 305 to obtain a clustered graph 402. The pruning component 403 can prune edges between nodes of the clustered graph belonging to different clusters to obtain refined graph 405. The output of the ascGTN 305 (e.g., imputed transcriptomics 306) can be generated by applying a graph transformer network from the ascGTN 305 to the refined graph 405.

The parameters of the ascGTN 305 can be trained jointly with the joint parameter training component 407. The joint parameter training component 407 can utilize variational optimization, gradient descent, and data-dependent loss functions.

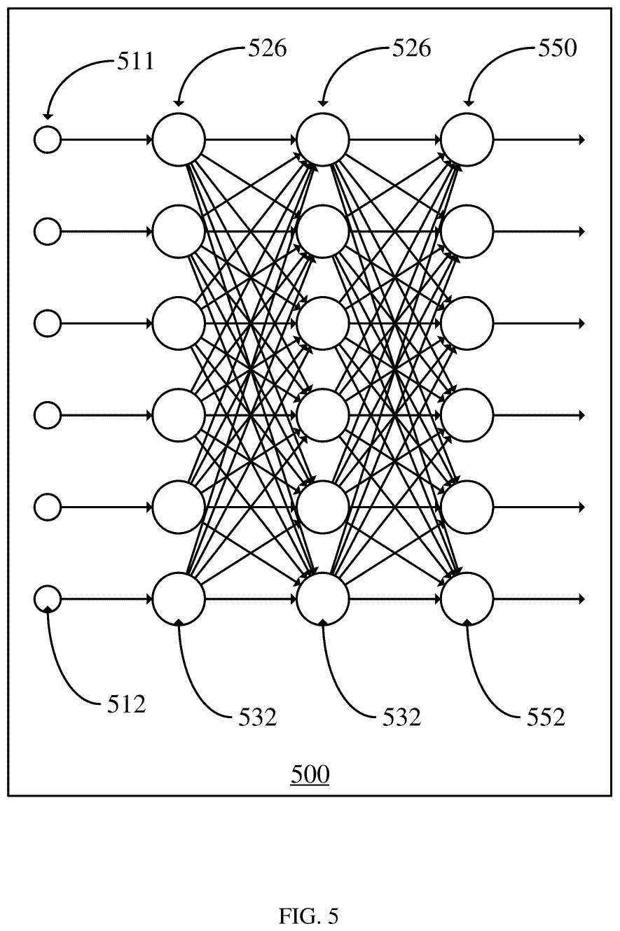

Referring now to FIG. 5, a block diagram shows a neural network for digital profiling of fine-grained spatial transcriptomics from images, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example can be associated with a known result or output. Each example can be represented as a pair, (x, y), where x represents the input data and y represents the known output. The input data can include a variety of different data types and can include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments can be made to the stored weights through back propagation, where the effect of the weights on the output values can be determined by calculating the mathematical gradient and adjusting the weights in a manner that shifts the output towards a minimum difference. This optimization, referred to as a gradient descent approach, is a non-limiting example of how training can be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 500, such as a multilayer perceptron, can have an input layer 511 of source neurons 512, one or more computation layer(s) 526 having one or more computation neurons 532, and an output layer 540, where there is a single output neuron 552 for each possible category into which the input example could be classified. An input layer 511 can have a number of source neurons 512 equal to the number of data values in the input data 511. The computation neurons 532 in the computation layer(s) 526 can also be referred to as hidden layers, because they are between the source neurons 512 and output neuron(s) 552 and are not directly observed. Each neuron 532, 552 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, w₂, . . . wₙ₋₁, wₙ. The output layer provides the overall response of the network to the inputted data. A deep neural network can be fully connected, where each neuron in a computational layer is connected to all other neurons in the previous layer, or can have other configurations of connections between layers. If links between neurons are missing, the network is referred to as partially connected.

In an embodiment, the ascGTN 305 architecture can use a GTN backbone with an extra layer of weights, W₀, parameterizing a linear projection function of the input features, which can create a vector of latent meta-features per compartment c and node n,

$${z}_{i n}^{c} = {x}_{i , n}^{c} {W}_{0} .$$

Training a deep neural network can involve two phases, a forward phase where the weights of each neuron are fixed and the input propagates through the network, and a backwards phase where an error value is propagated backwards through the network and weight values are updated. The computation neurons 532 in the one or more computation (hidden) layer(s) 526 perform a nonlinear transformation on the input data 512 that generates a feature space. The classes or categories can be more easily separated in the feature space than in the original data space.

In an embodiment, the ascGTN 305 can be trained to determine imputed transcriptomics from image data 104 by utilizing various loss functions that depend on the composition of image data 104. In another embodiment, the ascGTN 305 can be trained to learn the imputed spatial marker 117 for downstream tasks 120 based on the imputed transcriptomics based on input data for the downstream tasks 120.

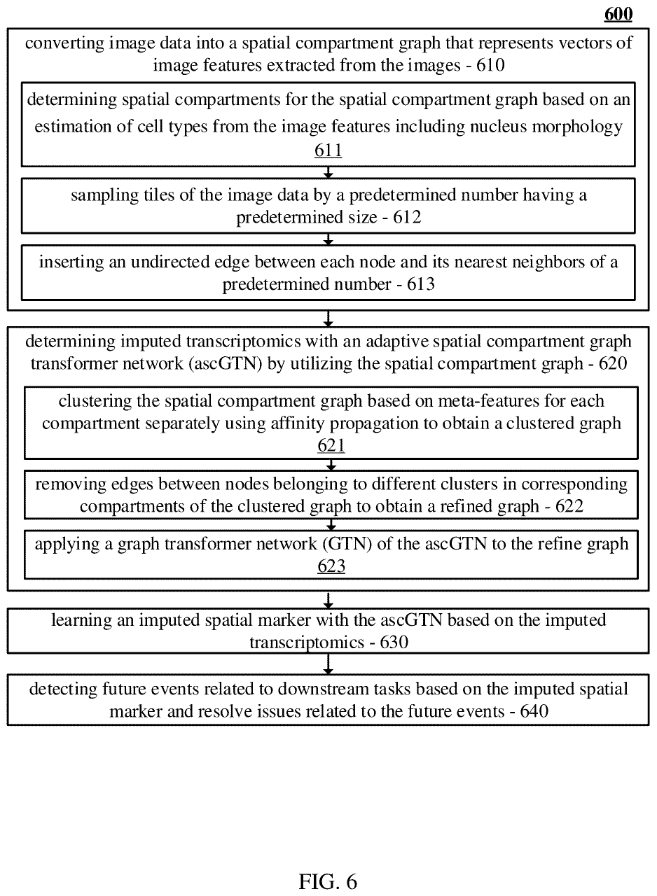

Referring now to FIG. 6, a flow diagram shows a high-level overview of a method for digital profiling of fine-grained spatial transcriptomics from images, in accordance with an embodiment of the present invention.

In an embodiment, image data can be converted into a spatial compartment graph that represents vectors of image features extracted from the images. Imputed transcriptomics can be determined with an adaptive spatial compartment graph transformer network (ascGTN) by utilizing the spatial compartment graph. An imputed spatial marker can be learned with the ascGTN based on the imputed transcriptomics. Future events related to downstream tasks can be detected based on the imputed spatial marker and resolve issues related to the future events.

In block 610, image data can be converted into a spatial compartment graph that represents vectors of image features extracted from the images.

In an embodiment related to cellular features, image data 104 associated with N_{data} slide images, where the data for image i∈{1 . . . N_{data}} can be represented by vectors of image features

$${x}_{in}^{c}$$

extracted from each image, where

$${x}_{i n}^{c}$$

is the vector associated with the c'th cellular compartment of the n'th tile associated with slide i, along with an edge matrix Eᵢ⊂{1 . . . Nᵢ}² representing which tiles are spatially adjacent in slide i. In another embodiment, image data can be obtained from a monitored entity 101 such as component 103.

The image data 104 can be formatted as a Spatial Compartment Graph (SCG) 303 for slide i, defined as

$${G}_{i} = \{ {X}_{i} , {E}_{i}^{SCG} \} .$$

### Here,

$${X}_{i} = [ {X}_{i}^{1} ; … ; {X}_{i}^{C} ] ,$$

where [.;.] denotes vertical concatenation of matrices, and

$${X}_{i}^{c}$$

is a matrix of size Nᵢ×Dᵢₘ, whose n'th row contains the image features from the c'th compartment of slide i, i.e.

$${x}_{i n}^{c} .$$

### Additionally,

$${E}_{i}^{SCG} ⊂ {( \{ 1 … {N}_{i} \} \times \{ 1 … C \} )}^{2} ,$$

where C is the number of cellular compartments, and we have

$$( {n}_{1} , {c}_{1} , {n}_{2} , {c}_{2} ) ∈ {E}_{i}^{SCG} iff ( {n}_{1} , {n}_{2} ) ∈ {E}_{i} ⋀ {c}_{1} = {c}_{2} ,$$

or n₁=n₂.

In block 611, for each tile, a nucleus segmentation engine can be used to detect meta-features such as predetermined structures like cellular nuclei, and extract image features such as nucleus morphological features for each cell to determine the spatial compartments. The nucleus segmentation engine classifies each nuclei into cell types (neoplasic, lymphocytes, macrophages, connective tissue etc.). Each cell type is treated as a separate spatial compartment, and the morphological features for all nuclei in tile n belonging to a given cell-type c are averaged to provide the image features

$${x}_{i n}^{c} .$$

In another embodiment, other sub-parts of the entity can be detected into corresponding types (e.g., component sub-parts, types, etc.) based on domain knowledge and a classifying component (e.g., segmentation model).

For compartments associated with no predetermined structure (e.g., nuclei) in a given tile,

$${x}_{i n}^{c}$$

is set to the 0 vector. Further, associated gene expression data corresponding to each slide, Yᵢ can be provided. Here, Yᵢ can be a vector of bulk expression values of length D_{struc}. Alternatively, Yᵢ can be a matrix of size Nᵢ×D_{struc}, whose n'th row represents the bulk expression of each gene in spatial tile n. Finally, Yᵢ can be a matrix of the form

$${Y}_{i} = [ {Y}_{i}^{1} ; … ; {Y}_{i}^{C} ] ,$$

where

$${Y}_{i}^{c}$$

is a matrix of size Nᵢ×D_{struc}, whose n'th row represents the expression of each gene in a type compartment c of spatial tile n.

Further details on how the SCG 303 can be obtained are described below.

In block 612, to form the SCG 303, tiles of the image data can be sampled by a predetermined number (e.g., 500) having a predetermined size (e.g., 512×512 pixels). The tiles can be positioned uniformly at random such that they do not overlap with previous tiles, and have an overlap of >90% with usable area (e.g., tissue, component part, etc.) of the slide.

In block 613, the SCG 303 (Et) can be formed by inserting an undirected edge between each node i, and its nearest neighbors of a predetermined number (e.g., k=6).

In block 620, imputed transcriptomics can be determined by the ascGTN 305 by utilizing the spatial compartment graph. With SCG 303, morphological image features, and training data, the ascGTN 305 architecture uses a GTN backbone with an extra layer of weights, W₀, which parameterizes a linear projection function of the input features. The ascGTN 305 can create a vector of latent meta-features per compartment c and node n,

$${z}_{i n}^{c} = {x}_{i , n}^{c} {W}_{0}$$

from the parameterization.

In block 621, for a given weight W₀, clustering can be performed by the clustering component 401 based on meta-features for each compartment separately using affinity propagation to obtain a clustered graph 402.

In block 622, the clustered graph 402 (e.g., graph Eᵢ) can be pruned by pruning component 403 by removing edges between nodes belonging to different clusters in the corresponding compartments to obtain the refined graph 405. The refined graph 405 can be defined as:

$${( {E}_{i}^{SCG} )}^{′} = \{ ( {n}_{1} , {c}_{1} , {n}_{2} , {c}_{2} ) \vert ( {n}_{1} , {c}_{1} , {n}_{2} , {c}_{2} ) ∈ {E}_{i}^{S C G} ⋀ ( {n}_{1} = {n}_{2} ⋁ ( {c}_{1} = {c}_{2} = c ⋀ {C}_{c} ( {n}_{1} ) = {C}_{c} ( {n}_{2} ) ) \} . \qquad ( 1 )$$

In block 623, the ascGTN output (e.g., imputed transcriptomics 306) can be generated by applying a graph transformer network (GTN) of the ascGTN 305 to the refined graph:

$$ascGTN ( {X}_{i} , {E}_{i}^{SCG} ; θ , {W}_{0} ) = GTN ( {{X}_{i} ( {E}_{i}^{SCG} )}^{′} ; θ ) , \qquad ( 2 )$$

where θ are the remaining parameters (e.g., weights and biases) of the GTN. The imputed transcriptomics 306 can include fine-grained details about a section of a monitored entity. In cellular applications, the imputed transcriptomics 306 can include cellular interactions, tissue architecture, disease mechanisms, etc.

In block 624, the weights W₀ and the remaining parameters of the GTN, which are denoted by θ, are then trained jointly, using Variational Optimization (on W₀) combined with gradient descent on the remaining parameters θ. The loss function can include various forms depending on the data (e.g., bulk, tile-level and compartment specific data) used, which can be combined if heterogenous data is used.

For bulk data, the mean square error (MSE) loss can be used. For tile and compartment specific data, the losses in Eqs. 3 and 4 are used respectively:

$$ℒ ( {X}_{i}^{L} , {Y}_{i} ) = MSE ( {X}_{i , g}^{L} , {Y}_{i} ) - λ {\sum }_{g} P C C ( {X}_{i , g}^{L} , {Y}_{i , g} ) , \qquad ( 3 )$$

$$ℒ ( {X}_{i}^{L} , {Y}_{i} ) = MSE ( {X}_{i}^{L} , {Y}_{i} ) - λ {\sum }_{g , c} PCC ( {X}_{i , g}^{c , L ,} , {Y}_{i , g}^{c} ) , \qquad ( 4 )$$

where λ is a scalar hyperparameter that controls the trade-off between the MSE and PCC losses which can be optimized using a validation set for optimal performance on downstream tasks, PCC is the Pearson correlation term, g is a gene.

The overall objective to jointly train the parameters of the ascGTN can include:

$${min}_{θ , {W}_{0}} {\sum }_{i} ℒ ( ascGTN ( {X}_{i} , {E}_{i}^{S C G} ; θ , {W}_{0} ) , {Y}_{i} ) , \qquad ( 5 )$$

If multiple types of expression data are available during training, the losses above can be combined, and the appropriate loss applied to datapoints from each source. In this case, alternative types of aggregation is applied at the networks final layer depending on the datatype, with no aggregation (i.e. the identity map) being applied in the case of compartment level expression data.

The ascGTN 305 can trained using bulk slide and/or compartment-specific expression data, where bulk data are used from a public source such as The Cancer Genome Atlas (TCGA), and experimental compartment specific data are used from Digital Spatial Profiling (DSP) of matching or neighboring slides of a tissue biopsy. The DSP data can generate expression associated with three compartments (tumor, lymphocytes, macrophages), which are matched to the corresponding compartments of the closest image tiles in the model. A Graph Transformer Network (GTN) can be trained using Gradient Descent, with the loss functions described herein.

The output of the ascGTN 305 (e.g., imputed transcriptomics) can be used to learn compartment specific spatial signatures for predicting future events related to the monitored entities 101.

In block 630, the imputed spatial marker can be learned based on the output of the ascGTN 305 (e.g., imputed transcriptomics 306). The outputs can be used as inputs to a Cox regression model to predict future events such as a patient's survival time, or a binary classifier to predict a response vs. resistance to treatment. In an embodiment to predict a response vs. resistance to treatment, response data can be obtained, Rᵢ∈{0,1} for a subset of patients, S_{clinical}, where 1 represents response and 0 represents resistance, for which image data also exists. Hence, the biomarker for response can be learned by optimizing the following objective:

$${min}_{β} {\sum }_{i ∈ {S}_{c l i n i c a l}} {ℒ}_{clinical} ( {β}^{T} \hat{{y}_{ι}} , {R}_{i} ) ,$$

$$where$$

$$\hat{{y}_{ι}} = ascGTN ( {X}_{i} , {E}_{i}^{S C G} ; θ , {W}_{0} ) ,$$

where θ is a hyperparameter of the GTN,

$$\hat{{y}_{ι}} = {X}_{i}^{L}$$

is assumed to be a vector of a predicted bulk expression, or a concatenation of the predicted compartment specific bulk expression vectors,

$$\hat{{y}_{ι}} = [ {X}_{i , c = 1}^{L} ; … ; {X}_{i , c = C}^{L} ] , : {X}_{i , c}^{L} = \frac{{\sum }_{n} {X}_{i , n , g}^{c , L - 1}}{{N}_{i}}$$

and β∈R is a vector of linear coefficients predicting the clinical outcome.

The loss, [symbol] , can be the cross entropy loss, to which a ridge, a least absolute shrinkage and selection operator (LASSO), or an elastic net regularization term can be added.

In block 640, the imputed spatial marker can be utilized to detect future events related to downstream tasks and resolve issues related to the future events.

In an embodiment, as discussed herein, the issues related to the future events depend on the downstream task being performed. For the downstream task of cancer treatment, the future events can be related to a response of a patient 102 to an ongoing treatment. An issue can include the negative response of patient 102 to the treatment and a resolution can include changing the treatment.

For the downstream task of anomaly detection, the future events can be related to operational parameter readings for the monitored component 103. The issue can include abnormal operational parameter readings (e.g., temperature, intensity, alignment, etc.) of the component 103. The resolution for the issue can include stopping/starting the component 103, resetting the component 103, updating the operational parameters of the component 103, etc.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This can be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art can implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method, comprising:
converting image data into a spatial compartment graph that represents vectors of image features extracted from the images;
determining imputed transcriptomics with an adaptive spatial compartment graph transformer network (ascGTN) by utilizing the spatial compartment graph;
learning an imputed spatial marker with the ascGTN based on the imputed transcriptomics; and
detecting future events related to downstream tasks based on the imputed spatial marker and resolve issues related to the future events.

2. The method of claim 1, wherein converting the image data further comprising determining spatial compartments for the spatial compartment graph based on an estimation of cell types from the image features including nucleus morphology.

3. The method of claim 1, wherein converting the image data further comprises sampling tiles of the image data by a predetermined number having a predetermined size.

4. The method of claim 1, wherein converting the image data further comprises inserting an undirected edge between each node and its nearest neighbors of a predetermined number.

5. The method of claim 1, wherein determining the imputed transcriptomics further comprises clustering the spatial compartment graph based on meta-features for each compartment separately using affinity propagation to obtain a clustered graph.

6. The method of claim 5, wherein determining the imputed transcriptomics further comprises removing edges between nodes belonging to different clusters in corresponding compartments of the clustered graph to obtain a refined graph.

7. The method of claim 6, wherein determining the imputed transcriptomics further comprises applying a graph transformer network (GTN) of the ascGTN to the refine graph.

8. The method of claim 1, wherein the downstream tasks further comprise assisting a decision making of a decision-making entity by determining a patient response to a treatment based on the imputed spatial marker through automated decision making.

9. A system, comprising:
a memory device;
one or more processor devices operatively coupled with the memory device to perform operations including:
converting image data into a spatial compartment graph that represents vectors of image features extracted from the images;
determining imputed transcriptomics with an adaptive spatial compartment graph transformer network (ascGTN) by utilizing the spatial compartment graph;
learning an imputed spatial marker with the ascGTN based on the imputed transcriptomics; and
detecting future events related to downstream tasks based on the imputed spatial marker and resolve issues related to the future events.

10. The system of claim 9, wherein converting the image data further comprising determining spatial compartments for the spatial compartment graph based on an estimation of cell types from the image features including nucleus morphology.

11. The system of claim 9, wherein converting the image data further comprises sampling tiles of the image data by a predetermined number having a predetermined size.

12. The system of claim 9, wherein converting the image data further comprises inserting an undirected edge between each node and its nearest neighbors of a predetermined number.

13. The system of claim 9, wherein determining the imputed transcriptomics further comprises clustering the spatial compartment graph based on meta-features for each compartment separately using affinity propagation to obtain a clustered graph.

14. The system of claim 13, wherein determining the imputed transcriptomics further comprises removing edges between nodes belonging to different clusters in corresponding compartments of the clustered graph to obtain a refined graph.

15. The system of claim 14, wherein determining the imputed transcriptomics further comprises applying a graph transformer network (GTN) of the ascGTN to the refine graph.

16. The system of claim 9, wherein the downstream tasks further comprise assisting a decision making of a decision-making entity by determining a patient response to a treatment based on the imputed spatial marker through automated decision making.

17. A non-transitory computer program product comprising a computer-readable storage medium including a program code, wherein the program code when executed on a computer causes the computer to perform operations including:
converting image data into a spatial compartment graph that represents vectors of image features extracted from the images;
determining imputed transcriptomics with an adaptive spatial compartment graph transformer network (ascGTN) by utilizing the spatial compartment graph;
learning an imputed spatial marker with the ascGTN based on the imputed transcriptomics; and
detecting future events related to downstream tasks based on the imputed spatial marker and resolve issues related to the future events.

18. The non-transitory computer program product of claim 17, wherein converting the image data further comprises inserting an undirected edge between each node and its nearest neighbors of a predetermined number.

19. The non-transitory computer program product of claim 17, wherein determining the imputed transcriptomics further comprises clustering the spatial compartment graph based on meta-features for each compartment separately using affinity propagation to obtain a clustered graph.

20. The non-transitory computer program product of claim 19, wherein determining the imputed transcriptomics further comprises removing edges between nodes belonging to different clusters in corresponding compartments of the clustered graph to obtain a refined graph.
